(19) United States
(12) Patent Application Publication
(10) Pub. No.: US 2017/0360569 A1
(43) Pub. Date: Dec. 21, 2017

(54) PARTIAL JOINT RESURFACING

(71) Applicant: ARTHREX, INC., Naples, FL (US)

(72) Inventors: Marc Stoll, Fort Myers, FL (US); Nick J.T. Metcalfe, Fort Myers Beach, FL (US)

(21) Appl. No.: 15/184,188

(22) Filed: Jun. 16, 2016

(51) Int. Cl. A61F 2/40

(52) U.S. Cl. CPC A61F 2/4081; A61F 2/4014; A61F 2310/00011

(57) ABSTRACT

A method of resurfacing a glenoid according to an according to an exemplary aspect of the present disclosure includes resurfacing a first section of a glenoid surface with and implant while leaving a second section of the glenoid surface uncovered by the implant.

### BACKGROUND

This disclosure relates to joint resurfacing components and methods.

Known shoulder joint resurfacing methods include resurfacing the entire articulating surface of the glenoid. Advancements in this field of technology are desirable, especially when they pertain to less invasive solutions that expand the continuum of patient care options.

### SUMMARY

This disclosure details exemplary joint resurfacing methods and components during surgical procedures, such as glenoid resurfacing procedures. The components include an implant for placement on one surface of a joint for articulation with another surface.

A method of resurfacing a glenoid according to an according to an exemplary aspect of the present disclosure includes resurfacing a first section of a glenoid surface with and implant while leaving a second section of the glenoid surface uncovered by the implant.

A method of resurfacing a glenoid according to an exemplary aspect of the present disclosure includes fixating an implant to a glenoid. The glenoid includes first and second concave surfaces, and the implant is fixated to only cover one of the first and second concave surfaces.

An implant for resurfacing a joint according to according to an exemplary aspect of the present disclosure includes an articulating surface component. A baseplate component is configured to receive the articulating surface component and includes a plurality of removable scores for customizing a size of the baseplate component.

The embodiments, examples and alternatives of the preceding paragraphs, the claims, or the following description and drawings, including any of their various aspects or respective individual features, may be taken independently or in any combination. Features described in connection with one embodiment are applicable to all embodiments, unless such features are incompatible.

The various features and advantages of this disclosure will become apparent to those skilled in the art from the following detailed description. The drawings that accompany the detailed description can be briefly described as follows.

### BRIEF DESCRIPTION OF THE DRAWINGS

FIG. 1 illustrates an example resurfaced joint. In this example, the resurfaced joint is a shoulder.

FIG. 2a illustrates a top view of a glenoid experiencing a first amount of deterioration.

FIG. 2b illustrates a top view of a glenoid experiencing a second amount of deterioration.

FIG. 3 illustrates an example resurfaced joint.

FIG. 4 illustrates another example resurfaced joint.

FIG. 5 illustrates a modular implant.

FIG. 6 illustrates a scored baseplate.

### DETAILED DESCRIPTION

This disclosure describes components and methods for joint resurfacing. For example, the components or methods could be used in glenoid resurfacing and/or a shoulder replacement procedure.

In some embodiments, the joint is a shoulder, and a partial surface of the glenoid of the shoulder is resurfaced with an implant. These and other features are described in greater detail in the following paragraphs of this detailed description.

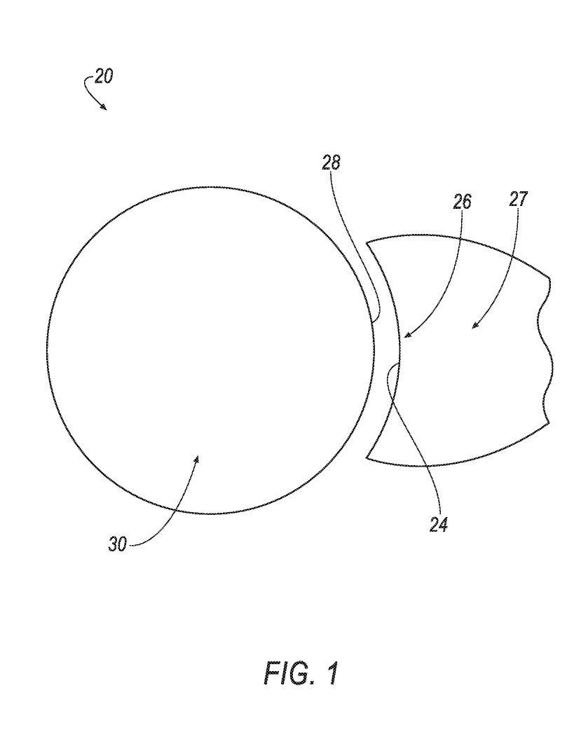

FIG. 1 illustrates an example joint 20. In this non-limiting example, the joint 20 is a shoulder. The joint 20 includes a surface 24 of a glenoid cavity (“glenoid”) 26 of a scapula 27. The surface 24 interfaces with an articulating surface 28 of a humerus 30. The humerus 30 could be a prosthetic humerus or the existing or native humerus of the patient. Over time, arthritis may develop and cause deterioration in all or part of the glenoid 26. The glenoid 26 may be resurfaced to repair the joint. Exemplary resurfacing techniques & devices are detailed below.

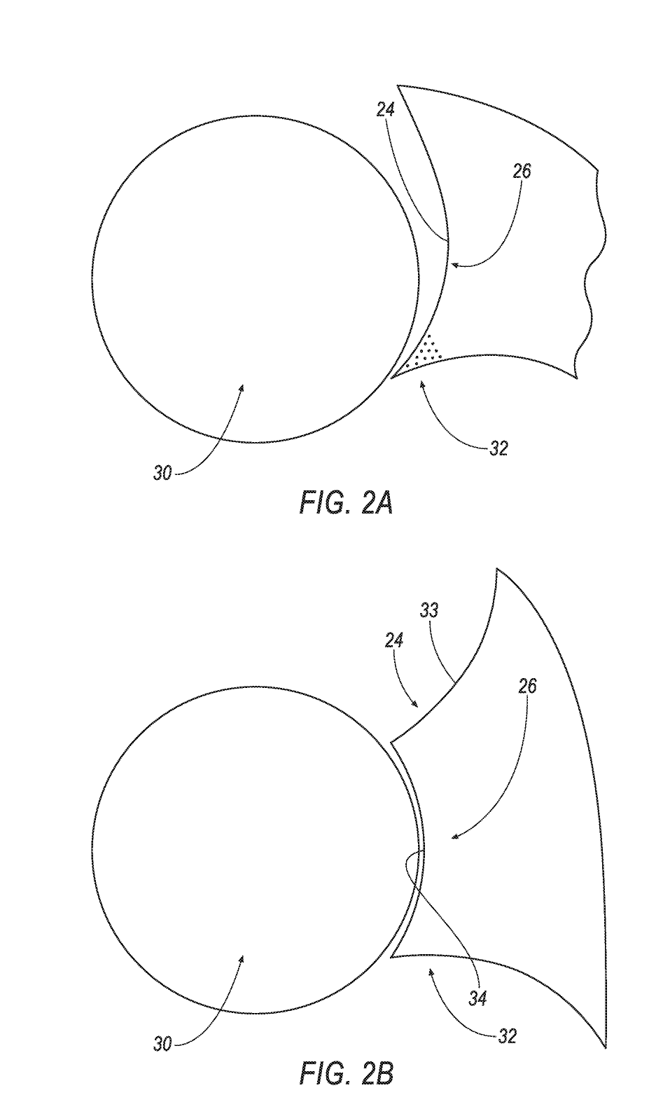

FIGS. 2a and 2b illustrate top views of example deterioration that could occur in the glenoid 26. In this pathological situation, the glenoid 26 is the native glenoid. Glenoid shapes in osteoarthritis include what may be referred to as B1 or B2 glenoids characterized by posterior humeral subluxation or malalignment. FIG. 2a illustrates what may be referred to as a B1 glenoid. In a B1 glenoid, the humerus 30 has subluxated toward the posterior section 32 of the glenoid 26, which may result in deterioration of the posterior section 32 of the glenoid 26.

FIG. 2b illustrates what may be referred to as a B2 glenoid. In a B2 glenoid, the humerus 30 has subluxated toward the posterior section 32 of the glenoid 26 such that a first concave surface 33 and a second concave surface 34 have formed in the glenoid surface 24. The surface 24 of a B2 glenoid is thus considered biconcave. A B1 glenoid may progress into a B2 glenoid.

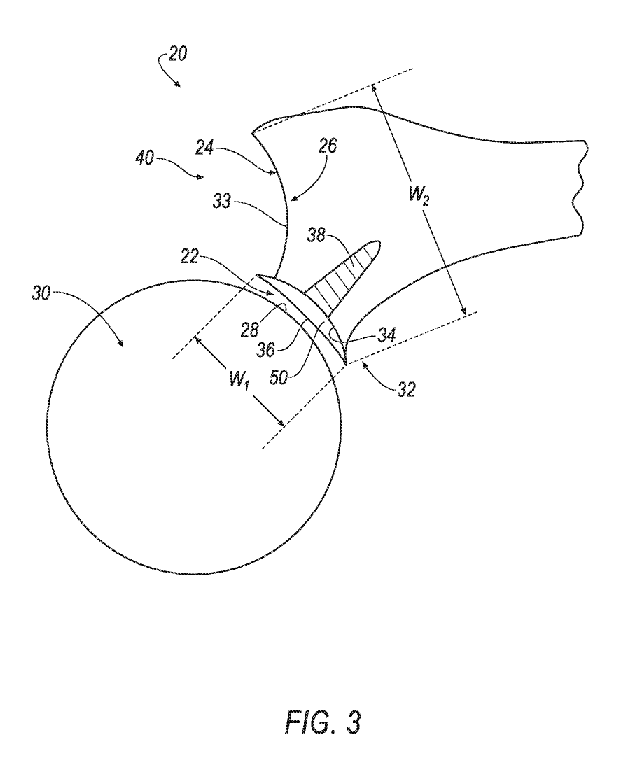

FIG. 3 illustrates a first example resurfacing of a glenoid 26. In this non-limiting example, the glenoid 26 is a B2 glenoid, and an implant 22 is fixated to the concave surface 34 at the posterior section 32 of the glenoid 26. The implant 22 includes an articulating surface 36 configured to interface with the articulating surface 28 of the humerus 30. In one non-limiting example, the articulating surface 36 of the implant 22 is made of plastic. In another non-limiting example, the articulating surface 36 of the implant 22 is made of a biologic material, such as bone, or allograft bone. The implant 22 further includes an attachment feature 38 configured for attachment to the glenoid 26. In one example, the attachment feature 38 is an elongated member configured for extension into the scapula 27. The attachment feature 38 may be a keel, peg, screw, or a plurality of cemented or cementless features to enhance fixation or rotational support to aide in fixation and rotation support.

In this non-limiting example, the posterior section 32 of the surface 24 of the glenoid 26 is resurfaced, while the anterior section 40 of the surface 24 of the glenoid 26 is not covered by the implant 22 and therefore not resurfaced. Thus, because the concave surface 34 articulates with the humerus 30 and the concave surface 33 does not, only a portion of the glenoid 26 is resurfaced with the implant 22. In this example, the humerus 30 is left to articulate in its non-anatomic position at the posterior section 32 of the glenoid 26. The width W1 of the articulating surface 36 of the implant 22 is less than the width W2 of the surface 24 of the glenoid 26, such that the surface area of the implant 22 is less than the surface area of the surface 24 of the glenoid 26.

Although the resurfaced section of the glenoid 26 in the example is the posterior section 32, the anterior section 40 could alternatively be the resurfaced section. In the case of a B2 glenoid, one of the concave surfaces 33, 34 is resurfaced, while the other of the concave surfaces 33, 34 is not resurfaced. In another example, the glenoid 26 may be partially resurfaced such that a central section of the glenoid 26 is resurfaced, while a peripheral section is not resurfaced.

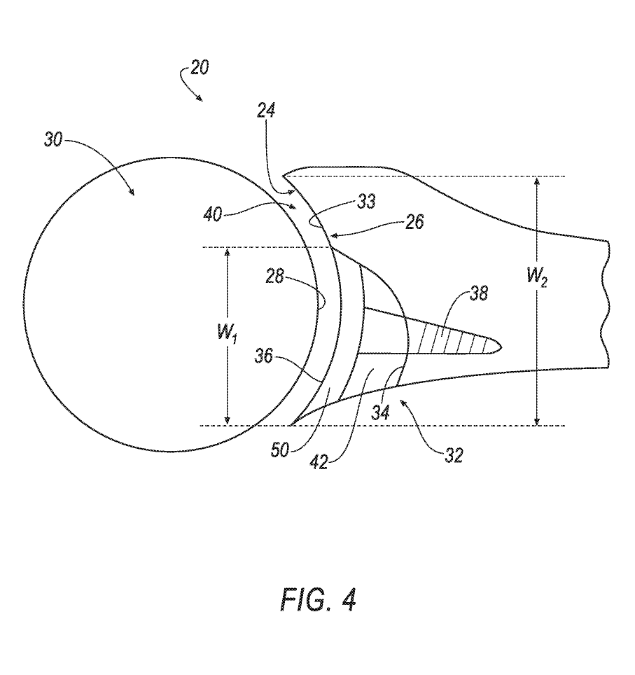

FIG. 4 illustrates a second example glenoid resurfacing. In this non-limiting example, the glenoid 26 is a B2 glenoid, and the glenoid 26 is partially resurfaced to return the humerus 30 to its anatomic position. An implant 22 is fixated at the concave surface 34 at the posterior section 32 of the glenoid 26. The implant 22 includes an articulating surface 36 configured to interface with the articulating surface 28 of the humerus 30. The implant 22 further includes an attachment feature 38 configured for attachment to the glenoid 26.

In one non-limiting example, a graft or augment 42 (biologic or metallic) may be located between the articulating surface 36 of the implant 22 and the concave surface 34. In addition to improving fixation, the graft 42 may space the articulating surface 36 from the surface 34 and orient the implant 22 and articulating surface 36 in such a way that the articulating surface 36 and the surface 33 create a single concave articulating surface either at the time of implantation or following an interfacing with the humerus 30.

In this non-limiting example, the concave surface 34 at the posterior section 32 of the surface 24 of the glenoid 26 is resurfaced, while the concave surface 33 at the anterior section 40 of the surface 24 of the glenoid 26 is not covered by the implant 22 and therefore not resurfaced. The articulating surface 36 of the implant 22 and the surface 33 create a single concave surface for the glenoid 26. The humerus 30 may then be re-centered into an anatomic position to articulate with both the implant 22 and the surface 33 of the partially resurfaced glenoid 26. The width W1 of the articulating surface 36 of the implant 22 is less than the width W2 of the surface 24 of the glenoid 26, such that the surface area of the implant 22 is less than the surface area of the surface 24 of the glenoid 26.

Although the resurfaced section of the glenoid 26 in the example is the posterior section 32, the anterior section 40 may be the resurfaced section. In the case of a B2 glenoid, one of the concave surfaces 33, 34 is resurfaced, while the other of the concave surfaces 33, 34 is not resurfaced.

The disclosed non-limiting methods thus describe partial joint resurfacing of a B2 glenoid to allow for articulation of the humeral head in an anatomic position by re-centering the humeral head or in a non-anatomic position by not re-centering the humeral head. The disclosed methods may be used to treat a B2 glenoid or to slow the progression of a B1 glenoid into a B2 glenoid or any such progressive pathology. The disclosed methods may also be used to treat other types of partially worn glenoids.

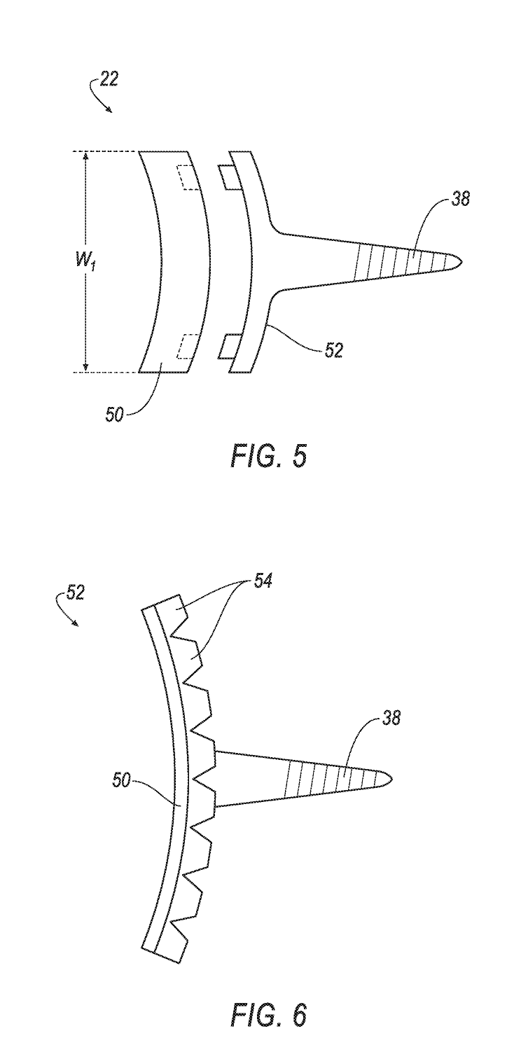

Referring to FIG. 5, the implant 22 may be modular in nature. The implant 22 may include an articulating surface component 50 that includes the articulating surface 36. In shoulder applications, for example, the width W1 of the articulating surface 36 of the implant 22 is less than the width of the surface of the glenoid 26. The articulating surface component 50 is configured for attachment to a baseplate component 52. The baseplate component 52 is configured for attachment to the resurfaced joint. In one non-limiting example, the articulating surface component 50 is made of plastic, one example being a polyethylene, and the baseplate component 52 is metallic. In another non-limiting example, the articulating surface component 50 is made of a biologic material, such as, but not limited to, bone or allograft bone.

FIG. 6 illustrates one example baseplate 52. The baseplate 52 may include scores 54 to allow for easy size customization to the resurfaced section of the joint, such as the glenoid in the shoulder. The baseplate 52 includes removable incremental scores 54 for customization of the size of the implant 22 to fit the partial surface of the joint. The scores 54 may be broken off to create a custom sized baseplate.

Another non-limiting example implant 22 may be an all biologic implant. The implant may be an allograft where bone and cartilage are implanted onto a defect to resurface a damaged glenoid 26.

Another non-limiting example implant 22 may be an all polyethylene implant. The polyethylene implant may be cemented into place. The polyethylene may include an attachment feature 38 such as a keel or peg to aide in fixation and rotation support.

Although the different non-limiting embodiments are illustrated as having specific components, the embodiments of this disclosure are not limited to those particular combinations. It is possible to use some of the components or features from any of the non-limiting embodiments in combination with features or components from any of the other non-limiting embodiments.

It should be understood that like reference numerals identify corresponding or similar elements throughout the several drawings. It should also be understood that although a particular component arrangement is disclosed and illustrated in these exemplary embodiments, other arrangements could also benefit from the teachings of this disclosure.

The foregoing description shall be interpreted as illustrative and not in any limiting sense. A worker of ordinary skill in the art would understand that certain modifications could come within the scope of this disclosure. For these reasons, the following claims should be studied to determine the true scope and content of this disclosure.

1. A method of resurfacing a glenoid, comprising:
resurfacing a first section of a glenoid surface with an implant;
leaving a second section of the glenoid surface uncovered by the implant;
positioning a humerus proximate to the implant, such that the humerus articulates with both the implant and the second surface.

2. (canceled)

3. The method as recited in claim 2, wherein the humerus is a biologic humerus.

4. The method as recited in claim 2, wherein the humerus is a prosthetic humerus.

5. The method as recited in claim 1, wherein the first section is at a posterior section of the glenoid.

6. The method as recited in claim 5, wherein the second section is at an anterior section of the glenoid.

7. The method as recited in claim 1, wherein a surface area of an articulating surface of the implant is less than a surface area of the glenoid.

8. The method as recited in claim 1, wherein the resurfaced first section and the second section provide a biconcave glenoid surface.

9. The method as recited in claim 1, wherein the resurfaced first section and the second section provide a single concave glenoid surface for articulation with the humerus.

10. The method as recited in claim 1, wherein the implant comprises a biologic material.

11. The method as recited in claim 1, wherein the implant comprises a plastic articulating surface and a metal baseplate.

12. A method of resurfacing a glenoid, comprising:
fixating an implant to a glenoid, the glenoid comprising first and second concave surfaces, wherein the implant is fixated to only cover one of the first and second concave surfaces; and
positioning a humerus proximate to the implant to articulate with the implant and the other of the first and second concave surfaces.

13. The method as recited in claim 12, wherein the implant covers the first concave surface at a posterior section of the glenoid.

14. The method as recited in claim 12, wherein the implant covers the second concave surface at an anterior section of the glenoid

15. The method as recited in claim 12, wherein the implant is oriented to resurface the first concave surface such that an articulating surface of the implant and the second concave surface establish a single concave articulating surface for articulating with the humerus.

16. An implant for resurfacing a joint comprising:
an articulating surface component; and
a baseplate component configured to receive the articulating surface component and including a plurality of removable scores for customizing a size of the baseplate component.

17. The implant as recited in claim 16, wherein the articulating surface component comprises a plastic material.

18. The implant as recited in claim 16, wherein the surface area of the articulating surface component is less than the surface area of the joint.

19. The implant as recited in claim 16, comprising an attachment feature configured to attach the implant to a surface of the joint.

20. The method as recited in claim 1, wherein the positioning includes re-centering the humerus in an anatomic position to articulate with the implant and the second section.

21. The method as recited in claim 12, wherein the positioning includes re-centering the humerus in an anatomic position to articulate with the implant and the second section.
